(19) United States
(12) Patent Application Publication
(10) Pub. No.: US 2013/0059343 A1
(43) Pub. Date: Mar. 7, 2013

(54) NUCLEOTIDE DERIVATIVES

(71) Applicant: Lael Cheung, Lincoln, NE (US)

(72) Inventor: Lael Cheung, Lincoln, NE (US)

(73) Assignee: LI-COR, INC., Lincoln, NE (US)

(21) Appl. No.: 13/570,204

(22) Filed: Aug. 8, 2012

Related U.S. Application Data
(60) Provisional application No. 61/531,568, filed on Sep. 6, 2011.

(51) Int. Cl. C07D 403/06; C07H 19/10; C12P 19/34; C07H 19/14

(52) U.S. Cl. 435/91.5; 548/427; 536/26.7; 536/26.8

(57) ABSTRACT

The present invention provides compounds and methods for attaching fluorescent labels to biological molecules such as nucleotides. The compounds and methods are useful for biological assays including DNA modification reactions.

### CROSS REFERENCE TO RELATED APPLICATION

The present application claims the benefit of priority to U.S. Provisional Application No. 61/531,568, filed Sep. 6, 2011, which is incorporated herein by reference in its entirety.

### BACKGROUND OF THE INVENTION

DNA sequencing is an important analytical technique in molecular biology. The development of sequencing techniques has led to advances in both the analysis and manipulation of genetic material. Well-known methods of DNA sequencing include the Maxam-Gilbert chemical degradation method, described in Maxam et al., Meth. in Enzym. 65:499 (1980), and the Sanger dideoxy chain termination technique, described in Sanger et al., P.N.A.S. USA 74:5463 (1977). In each method, DNA fragments labeled with ³²P are generated, which are then analyzed by gel electrophoresis. Both methods are useful, although they can prove to be difficult and slow.

As a result, other methods have been sought, including those that do not rely upon short-lived radioisotopes, such as ³²P. Several alternative methods of detection have been developed based on fluorescent labels. Next generation sequencing techniques include single molecule sequencing as disclosed by Williams in U.S. Pat. No. 6,255,083. In those methods, fluorescently labeled phosphate moieties are detected as they are released as pyrophosphates while a polymerase extension product is created.

In addition, U.S. Pat. No. 6,027,709 to Little et al., discloses phosphoramidite derivatives of cyanine dyes. The phosphoramidite derivatives are useful in labeling nucleotides that can serve as chain terminators in DNA synthesis techniques.

Connection (or ligation) of two fragments to make a larger molecule or structure is often achieved with the help of so-called “click chemistry” described by Sharpless et al. Angew. Chem., Int. Ed. 40: 2004 (2001). This term is used to describe a set of bimolecular reactions between two different reactants such as azides and acetylenes. The formation of 1,2,3-triazoles in 1,3-dipolar cycloaddition of azides to a triple bond is known, but because the activation energy of acetylene-azide cycloaddition is relatively high, the reaction is slow under ambient conditions.

The utility of the reaction of azides with alkynes was expanded by the discovery of Cu (I) catalysis. 1,3-cycloaddition of azides to terminal acetylenes in the presence of catalytic amounts of cuprous salts is facile at room temperature in organic or aqueous solutions.

U.S. Pat. No. 7,807,619 to Bertozzi et al. teaches modified cycloalkyne compounds and method of use of such compounds in modifying biomolecules. Bertozzi et al. teach a cycloaddition reaction that can be carried out under physiological conditions. As disclosed therein, a modified cycloalkyne is reacted with an azide moiety on a target biomolecule, generating a covalently modified biomolecule.

Despite the advances in nucleic acid sequencing and click chemistry techniques, there is a need for nucleotides with fluorescent labels attached, which are useful for biological assays including DNA synthesis reactions. The present invention satisfies this and other needs.

### BRIEF SUMMARY OF THE INVENTION

The present invention provides cyanine dyes with click chemistry functionalities useful for labeling biomolecules. As such, in one aspect, the present invention provides compounds of Formula I:

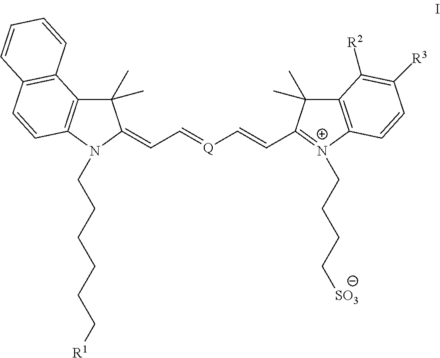

R¹ is a member such as an azido, C₂₋alkynyl, a pegylated azido and a pegylated C₂₋alkynyl;

R² and R³ are the same or different and wherein each is selected from the group of H, cyano, halo, trifluoromethylsulfonyl, sulfonato, or alternatively, join to form a six membered ring;

Q, if present is

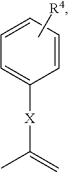

wherein X is a bond or heteroatom; and

R⁴ is a member selected from the group consisting of an azido, a C₂₋alkynyl, a pegylated azido and a pegylated C₂₋alkynyl.

In certain other aspects, the dyes of Formula I can be used to make nucleic acid chain terminators (dideoxy ribose nucleotides), wherein the dyes have been attached. As such, in another embodiment, the present invention provides compounds of Formula IIa-b, IIIa-b, IVa-b, and Va-b as defined herein.

In yet other aspects, the present invention relates to two components that interact with each other to form a stable covalent bio-orthogonal bond. Bio-orthogonal reactions are reactions of materials with each other, wherein each material has limited or essentially no reactivity with functional groups found in vivo. These components are of use in chemical and biological assays, as chemical reagents, medical imaging and therapy, and more particularly, in nucleic acid modification techniques. According to a particular embodiment of the invention, the covalent bio-orthogonal bond is obtained by the [3+2] cycloaddition of azides and alkynes.

In still other aspects, one of the two components that interact with each other to form a stable covalent bio-orthogonal bond is a near infrared dye, such as a cyanine dye. In a preferred aspect, the cyanine dyes of the present invention comprise either an azide or an alkyne group for use as a reactant in a click chemistry reaction and the other reactant is a biomolecule such as a nucleotide comprising either an alkyne or azide group.

These and other aspects, embodiments and advantages will become more apparent when read with the following detailed description.

### DETAILED DESCRIPTION OF THE INVENTION

### I. Definitions

The terms “a,” “an,” or “the” as used herein not only include aspects with one member, but also include aspects with more than one member. For example, an embodiment of a method of imaging that comprises using a compound set forth in claim 1 would include an aspect in which the method comprises using two or more compounds set forth in claim 1.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the quantity “X” only allows whole-integer values (e.g., “X carbons”) and X is at most 15, “about X” indicates from (X−1) to (X+1). In this case, “about X” as used herein specifically indicates at least the values X, X−1, and X+1. If X is at least 16, the values of 0.90X and 1.10X are rounded to the nearest whole-integer values to define the boundaries of the range.

When the modifier “about” is applied to describe the beginning of a numerical range, it applies to both ends of the range. Thus, “from about 700 to 850 nm” is equivalent to “from about 700 nm to about 850 nm.” When “about” is applied to describe the first value of a set of values, it applies to all values in that set. Thus, “about 680, 700, or 750 nm” is equivalent to “about 680 nm, about 700 nm, or about 750 nm.” However, when the modifier “about” is applied to describe only the end of the range or only a later value in the set of values, it applies only to that value or that end of the range. Thus, the range “about 2 to about 10” is the same as “about 2 to about 10,” but the range “2 to about 10” is not.

The term “activated alkyne,” as used herein, includes a chemical group that selectively reacts with an alkyne-reactive group, such as an azido group or a sulfhydryl group, on another molecule to form a covalent chemical bond between the activated alkyne group and the alkyne reactive group. As used herein activated alkyne encompasses any terminal alkynes or cyclic alkynes (dipolarophiles) that will react with 1,3-dipoles such as azides in a facile fashion.

The term “alkyne-reactive groups” include azide groups. Alkyne-reactive groups can also include a molecule that contains a chemical group that selectively reacts with an alkyne group.

“Azide” or “Azido” group as used herein includes a compound with a N₃ functional group. In certain aspects, 1,3-dipole-functional compounds can be used in the present invention which include, but are not limited to, azide-functional compounds, nitrile oxide-functional compounds, nitrone-functional compounds, azoxy-functional compounds, and/or acyl diazo-functional compounds.

“Azide reactive,” as used herein includes a material that selectively reacts with an azido group (N₃) on another molecule to form a covalent chemical bond between the azido group and the azide reactive group. Examples of azide-reactive groups include alkynes and phosphines (e.g., triaryl phosphine). “Azide-reactive” groups include a molecule that selectively reacts with an azido group.

“Cyanine dye” as used herein includes a compound having two substituted or unsubstituted nitrogen-containing heterocyclic rings joined by an unsaturated bridge.

“Halo” or “halogen” as used herein includes fluoro, chloro, bromo, or iodo.

“Heteroatom” as used herein includes an atom other than carbon or hydrogen. Representative heteroatoms include O, S, and N. The nitrogen or sulphur atom of the heteroatom is optionally oxidized to the corresponding N-oxide, S-oxide (sulfoxide), or S,S-dioxide (sulfone). In a preferred aspect, a heteroatom has at least two bonds to alkylene carbon atoms (e.g., —C₁-C₉ alkylene-O—C₁-C₉ alkylene-). In some embodiments, a heteroatom is further substituted with an acyl, alkyl, aryl, cycloalkyl, heterocyclyl, or heteroaryl group (e.g., —N(Me)—; —N(Ac)—).

When any two substituent groups or any two instances of the same substituent group are “independently selected” from a list of alternatives, they may be the same or different. For example, if R^{a} and R^{b} are independently selected from the group consisting of methyl, hydroxymethyl, ethyl, hydroxyethyl, and propyl, then a molecule with two R^{a} groups and two R^{b} groups could have all groups be methyl. Alternatively, the first R^{a} could be methyl, the second R^{a} could be ethyl, the first R^{b} could be propyl, and the second R^{b} could be hydroxymethyl (or any other substituents taken from the group). Alternatively, both R^{a} and the first R^{b} could be ethyl, while the second R^{b} could be hydroxymethyl (i.e., some pairs of substituent groups may be the same, while other pairs may be different).

“Phosphoramidityl” as used herein includes a trivalent phosphorous atom bonded to two alkoxy groups and an amino group.

In general, the unit prefix “u” as used herein is equivalent to “μ” or “micro.” For example, “ul” is equivalent to “μl” or “microliters.”

### II. Embodiments

A. Cyanine Dyes

A variety of cyanine dyes such as near-infrared cyanine dyes are suitable for use in the present invention. Such dyes include, dyes disclosed in U.S. Pat. No. 6,027,709, assigned to the present assignee and incorporated hereby reference in its entirety for all purposes.

More particularly, suitable cyanine dyes include for example compounds of Formula I:

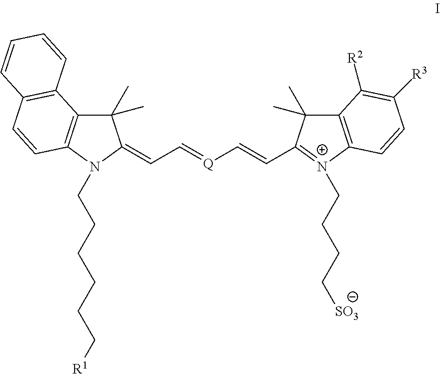

R¹ is a member such as an azido, a linker having an azido moiety, C₂₋alkynyl, a pegylated azido and a pegylated C₂₋alkynyl;

R² and R³ are the same or different and wherein each selected from the group of H, cyano, halo, trifluoromethylsulfonyl, sulfonato, or alternatively, join to form a six membered ring;

Q, if present is

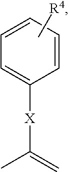

wherein X is a bond or heteroatom; and

R⁴ is a member selected from the group consisting of an azido, a C₂₋alkynyl, a pegylated azido and a pegylated C₂₋alkynyl.

Preferred dyes of Formula I include the following compounds in Table I:

TABLE I

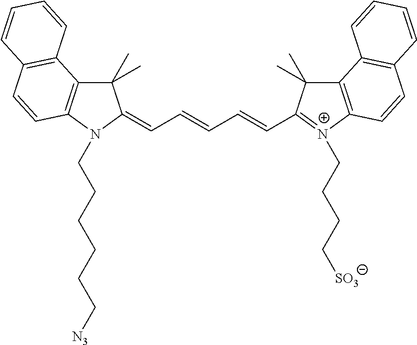

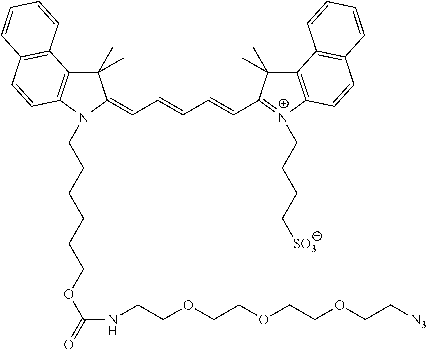

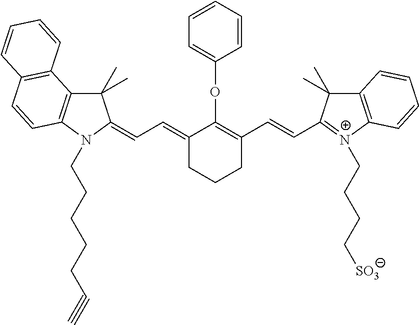

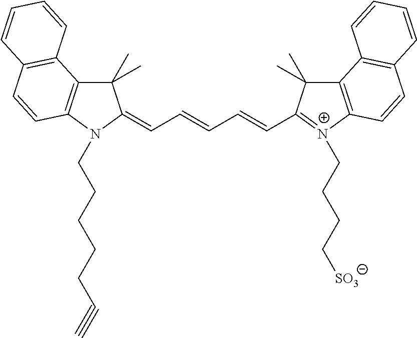

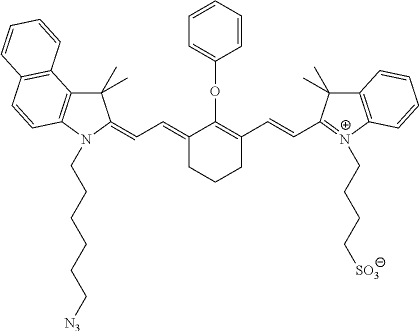

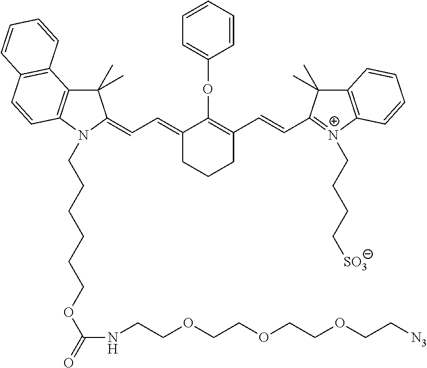

In certain other aspects, suitable cyanine dyes of the present invention include nucleic acid chain terminators (dideoxy ribose nucleotides), wherein the dyes have been attached. In another embodiment, the present invention provides compounds of Formula IIa-b, IIIa-b, IVa-b, and Va-b as follows, wherein R² and R³ are the same or different and wherein each is a member selected from the group of H, cyano, halo, trifluoromethylsulfonyl, sulfonato, or alternatively, join to form a six membered ring. Table II contains preferred chain terminators of the present invention.

TABLE II

| | |
|---|---|
| 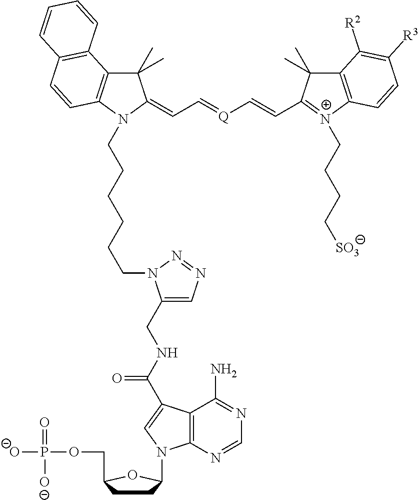 | Formula IIa-b |
| 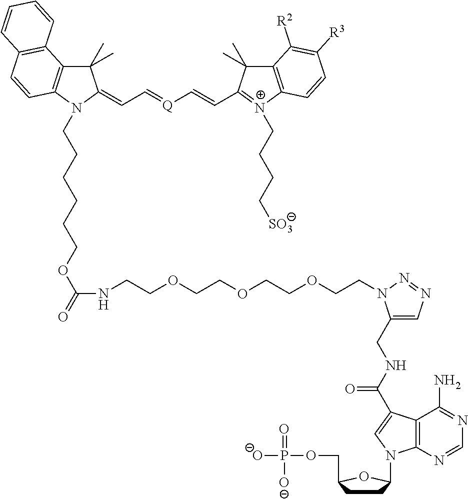 | |
| 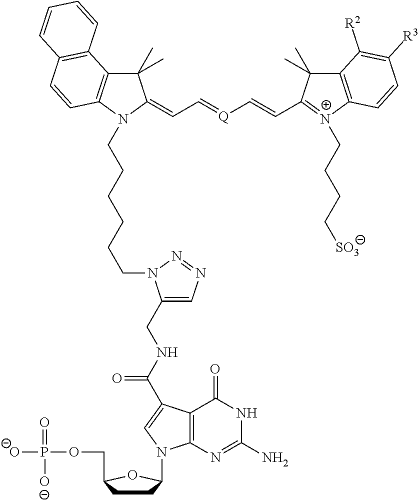 | Formula IIIa-b |
| 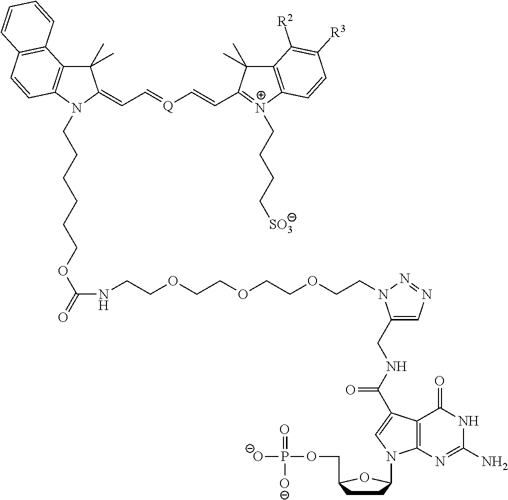 | |
| 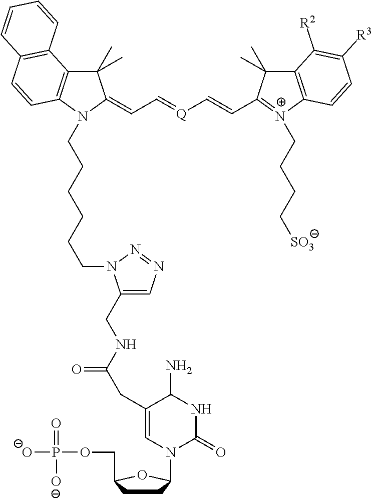 | Formula IVa-b |
| 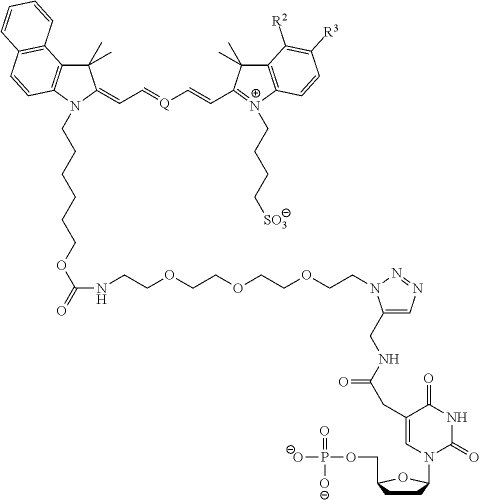 | |
| | Formula Va-b |
| 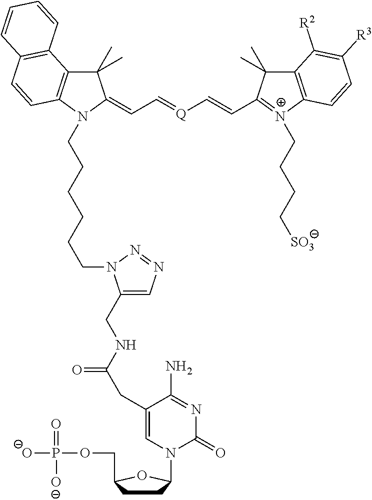 | |
| 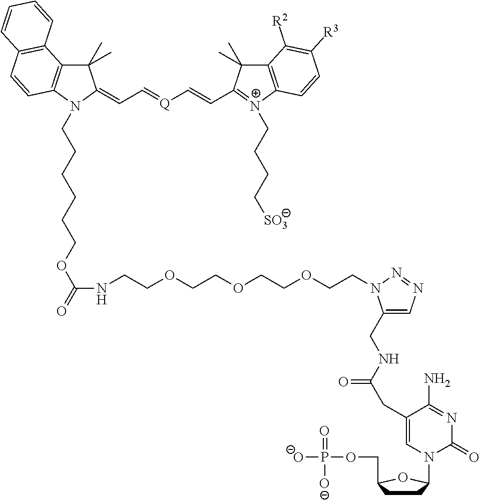 | |

The fluorescent dye-labeled DNA chain terminators are employed for the generation of fluorescent dye-labeled DNA sequencing fragments. A photometric detection system detects the fragments separated by electrophoresis. Fluorescence detection allows one to either scan a gel containing spatially resolved bands (i.e., resolution in space) or to sit at a single point on the gel and detect bands as they pass sequentially through the detection zone (i.e., resolution in time).

In another embodiment, the present invention provides a method for nucleic acid synthesis, said method comprising: reacting a polymerase, a nucleic acid template, a primer, dNTPs, and other reagents necessary for polymerization; and incorporating a chain terminator selected from the group consisting of Formula IIa-b, IIIa-b, IVa-b, Va-b and mixtures thereof, whereby nucleic acid synthesis ceases.

B. Methods of Making

The dyes of this invention wherein R¹ is —CO₂H or —OH can be synthesized by reacting the appropriate N-(carboxyalkyl)- or N-(hydroxyalkyl)-1,1,2-trimethyl-1H-benz(e)indolinium halide, preferably bromide, with sulfonatobutyl-1,1,2-trimethyl-1H-benz(e)indole at a relative molar ratio of about 0.9:1 to about 1:0.9, preferably 1:1 in an organic solvent, such as pyridine, and heated to reflux, followed by the addition of 1,3,3-trimethoxypropene in a relative molar ratio of about 1:1 to about 3:1 to the reaction product and continued reflux. The mixture subsequently is cooled and poured into an organic solvent such as diethyl ether. The resulting solid or semi-solid can be purified by chromatography on a silica gel column using a series of methanol/chloroform solvents.

Thereafter, it is possible to install an azido group into a cyanine dye represented by R—OH as is shown in Scheme I. The R—OH can be any dye containing entity, regardless of the point of attachment in relation to the chromophore.

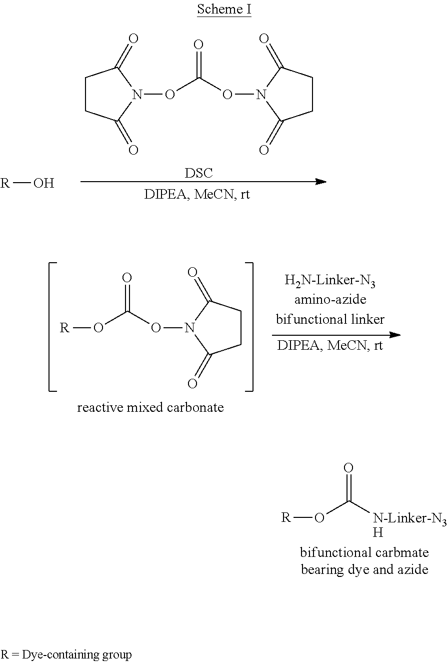

In Scheme I, a cyanine dye is added to a reaction solution of N,N′-disuccinimidyl carbonate and a molar excess of N,N-diisopropylethylamine in a reaction solvent (e.g., acetonitrile). The reaction is allowed to stir at ambient temperature, during which a reactive mixed carbonate intermediate is formed. Next, a solution of a bifunctional linker such as 11-azido-3,6,9-trioxaundecan-1-amine is added in and the reaction is stirred to generate, for example, a bifunctional carbamate having the dye on one end and the azide group on the other. Suitable linkers include, but are not limited to, polymers such as PEGs of varying lengths, monomeric and polymeric derivatives of nucleotides, carbohydrates, amino acids, lipids, glycols, alkanes, alkenes, arene, silicates, as well as biologically active and inactive compounds obtained from nature or from artificial synthesis.

The dyes of this invention have sufficient solubility in aqueous solutions that once they are attached to a soluble biomolecule, the biomolecule retains its solubility. They also have good solubility in organic media, which provides considerable versatility in synthetic approaches to the labeling of desired materials.

C. Click Functionalities

Azide reactive groups such as an alkyne compounds can react with at least one 1,3-dipole-functional compound such as an alkyne reactive group (e.g., a azido group) in a cyclization reaction to form a heterocyclic compound. In certain embodiments, the reaction can be carried out in the presence of an added catalyst (e.g., Cu(I)). In other embodiments, the reaction is carried out in the absence of such catalysts. Exemplary 1,3-dipole-functional compounds include, but are not limited to, azide-functional compounds, nitrile oxide-functional compounds, nitrone-functional compounds, azoxy-functional compounds, and/or acyl diazo-functional compounds. Preferably, azide-functional compounds are used.

In the [3+2] cycloaddition, an azide reacts with an alkyne to form a triazole adduct. This reaction can take place with a catalyst such as a Cu catalyst, or if the alkyne has enough strain (e.g. cycloalkyne ring) without a catalyst. Also for linear alkynes it is known that the reaction can take place without a catalyst, albeit at elevated temperatures. Reactions between azide and linear alkynes are for example described in Z. Li, T. Seok Seoa, J. Jua, Tetrahedron Letters 45 (2004) 3143-3146.

Scheme II shows a generalized synthetic scheme for one embodiment, which uses a catalyst such as a copper-catalyst. In Scheme II, the bifunctional carbamate having the dye of Formula I on one end and an azide group on the other is added to a alkyne derivative with copper(I) iodide in a reaction solvent to yield the [3+2] cycloaddition product.

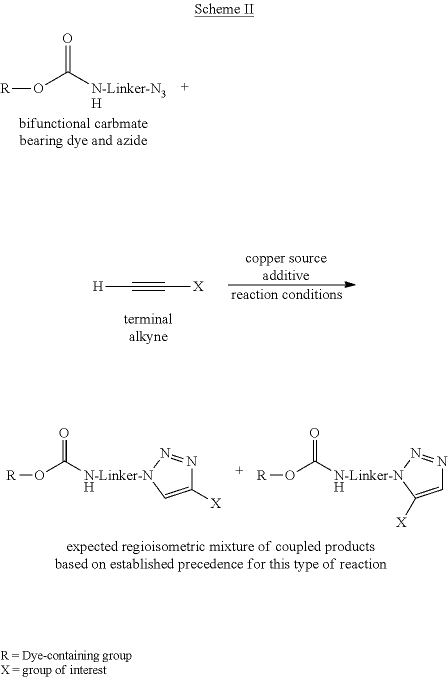

Suitable X moieties include, for example, monomeric and polymeric derivatives of nucleotides, carbohydrates, amino acids, lipids, glycols, alkanes, alkenes, arene, silicates, as well as biologically active and inactive compounds obtained from nature or from artificial synthesis.

As illustrations of the uses of the present invention, the compounds of this invention can be used as labeling reagents for analytical determination of proteins or for automated sequencing of DNA. Standard sequencing methodologies performed with labeled primers can produce high quality sequencing ladders and accurate DNA sequence data.

The compounds of this invention can be attached, for example, to analogs of nucleotide triphosphates (dNTPs and ddNTPs) to provide a reagent for enzymatic labeling of various DNA molecules and for facilitating their detection with an automated DNA sequencing and analysis system. DNA sequencing reaction products can be labeled internally by performing limited polymerization utilizing the labeled dNTP as the sole source of a particular deoxynucleotide prior to a dideoxy-specific termination reaction. PCR products also can be labeled fluorescently by the addition of limited quantities of the labeled dNTP to the amplification reaction. Such labeling can be useful, for example, for the detection of short tandem repeat polymorphisms (STRPs), which in turn are useful for gene mapping, genetic diagnostics, forensic analyses and paternity testing.

D. Biological Molecules

Other suitable biological molecules include those having a azido or alkynyl functionality, which include, but are not limited to, an antibody, an antigen, an avidin, a carbohydrate, a deoxy nucleic acid, a dideoxy nucleotide triphosphate, an enzyme cofactor, an enzyme substrate, a fragment of DNA, a fragment of RNA, a hapten, a hormone, a nucleic acid, a nucleotide, a nucleotide triphosphate, a nucleotide phosphate, a nucleotide polyphosphate, an oligosaccharide, a peptide, PNA, a polysaccharide, a protein, a streptavidin, and the like. These biological molecules will in turn be reacted with the dye compounds of the present invention comprising either an azide or an alkyne group for use in click chemistry reactions.

In more preferred aspects, the X moieties in Scheme II include an antibody, an avidin, and a streptavidin. Even more preferred aspects include a goat anti-mouse (GAM) antibody, a goat anti-rabbit (GAR) antibody, and streptavidin.

In certain other aspects, preferred X moieties include, but are not limited to, somatostatin, endostatin, a carbohydrate, an oligosaccharide, an aptamer, a liposome, PEG, an angiopoietin, angiostatin, angiotensin II, α₂-antiplasmin, annexin V, β-cyclodextrin tetradecasulfate, endoglin, endosialin, endostatin, epidermal growth factor, fibrin, fibrinopeptide β, fibroblast growth factor, FGF-3, basic fibronectin, fumagillin, heparin, hepatocycle growth factor, hyaluronan, an insulin-like growth factor, an interferon-α, β inhibitor, IL inhibitor, laminin, leukemia inhibitory factor, linomide, a metalloproteinase, a metalloproteinase inhibitor, an antibody, an antibody fragment, an acyclic RGD peptide, a cyclic RGD peptide, placental growth factor, placental proliferin-related protein, plasminogen, plasminogen activator, plasminogen activator inhibitor-1, a platelet activating factor antagonist, platelet-derived growth factor, a platelet-derived growth factor receptor, pleiotropin, proliferin, proliferin-related protein, a selectin, SPARC, a snake venom, substance P, suramin, a tissue inhibitor of a metalloproteinase, thalidomide, thrombin, thrombin-receptor-activating tetradecapeptide, transformin growth factor-α, β, transforming growth factor receptor, tumor growth factor-α, tumor necrosis factor, vitronectin, and the like.

In still other aspects, preferred X moieties include a carbohydrate and a carbohydrate derivative. Representative examples include glucosamine, a glyceraldehyde, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, and a derivative thereof. Even more preferred biomolecules include 2-deoxy-D-glucose, 2-deoxy-L-glucose, and racemic 2-deoxyglucose.

In yet still other aspects, the X moiety can be a ligand that has affinity for a receptor selected from the group of EGFR, Her2, PDGFR, IGFR, c-Ryk, c-Kit, CD24, integrins, FGFR, KFGR, VEGFR, TRAIL decoy receptors, retinoid receptor, growth receptor, PPAR, vitamin receptor, glucocorticosteroid receptor, Retinoid-X receptor, RHAMM, high affinity folate receptors, Met receptor, estrogen receptor and Ki67. Preferably, the biomolecule is a ligand that has affinity for an integrin receptor.

Alternatively, X is selected from the group of somatostatin, endostatin, a carbohydrate, a monosaccharide, a disaccharide, a trisaccharide, an oligosaccharide, aptamer, liposome and polyethylene glycol.

In yet another aspect, X is a small-molecule drug or drug-like molecule such as a tetracycline antibiotic, a tetracycline derivative, and calcein.

Alternatively, X is a small-molecule drug or peptide.

### III. Methods of Use

The azido or alkynyl functionalized dye compounds of the present invention provide click chemistry linking groups for attachment to a wide variety of biologically important molecules, including proteins, peptides, enzyme substrates, hormones, antibodies, antigens, haptens, avidin, streptavidin, carbohydrates, oligosaccharides, polysaccharides, nucleic acids, deoxy nucleic acids, fragments of DNA or RNA, cells and synthetic combinations of biological fragments such as peptide nucleic acids (PNAs). The dyes of this invention have sufficient solubility in aqueous solutions that once they are attached to a soluble biomolecule, the biomolecule retains its solubility. They also have good solubility in organic media, which provides considerable versatility in synthetic approaches to the labeling of desired materials. The compounds of the invention are useful in dry (i.e., water-free) conditions such as dry acetonitrile.

The dye compounds can have either an alkyne group or an azido group, which can then be used to link a biomolecule, such as DNA or RNA, through click chemistry. Use of click chemistry allows labeling of the DNA or RNA during the synthesis process. For example, the protected nucleotide is labeled while attached to a solid phase support. The dye labeled nucleotide having an azido group is reacted with a growing DNA or RNA strand having an alkynyl moiety. Alternatively, the dye labeled nucleotide having an alkynyl group is reacted with a growing DNA or RNA strand having an azido moiety. The labeled DNA or RNA is then cleaved from the solid phase using standardized procedures.

As such, in one embodiment, the present invention provides a method for nucleic acid synthesis, said method comprising: reacting a polymerase, a nucleic acid template, a primer, dNTPs, and other reagents necessary for polymerization; and incorporating a chain terminator selected from the group consisting of Formulas IIa-b, IIIc-b, IVa-b, Va-b and mixtures thereof, whereby nucleic acid synthesis ceases.

The present invention is illustrated below by the following examples. These examples are provided for illustrative purposes only and are not intended to be construed as limiting the scope of the invention.

### IV. Examples

Example 1 illustrates the synthesis of 4-(2-((E)-2-((E)-3-((E)-2-(3-(1-azido-13-oxo-3,6,9,14-tetraoxa-12-azaicosan-20-yl)-1,1-dimethyl-1H-benzo[e]indol-2(3H)-ylidene)ethylidene)-2-phenoxycyclohex-1-enyl)vinyl)-3,3-dimethyl-3H-indolium-1-yl)butane-1-sulfonate (“Compound 1”).

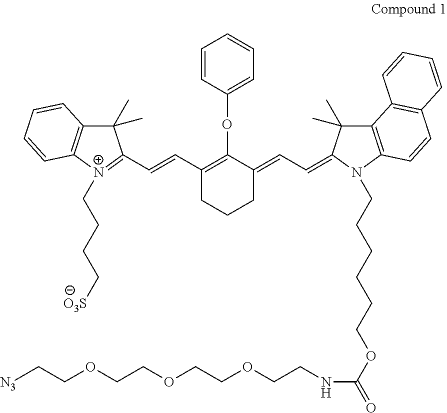

To a vigorously stirred solution of N,N′-disuccinimidyl carbonate (15.0 mg, 0.059 mmol) and N,N-diisopropylethylamine (0.020 mL, 0.12 mmol) in anhydrous acetonitrile (3.0 mL) was added 4-(2-((E)-2-((E)-3-((E)-2-(3-(6-hydroxyhexyl)-1,1-dimethyl-1H-benzo[e]indol-2(3H)-ylidene)ethylidene)-2-phenoxycyclohex-1-enyl)vinyl)-3,3-dimethyl-3H-indolium-1-yl)butane-1-sulfonate (20.0 mg, 0.025 mmol) in one portion. The reaction was allowed to stir at ambient temperature for 12 hours, during which the presumed mixed carbonate intermediate had formed (as determined by HPLC analysis). A solution of 11-azido-3,6,9-trioxaundecan-1-amine (15.0 mg, 0.069 mmol) in anhydrous acetonitrile (0.5 mL) was added in one portion and the reaction was allowed to stir at ambient temperature for an additional 2 hours. After HPLC analysis showed complete consumption of the presumed mixed carbonate intermediate, the reaction mixture was concentrated in vacuo to afford a crude residue. The residue was purified by reverse-phase flash chromatography to furnish the desired product IR800-PEG-Azide as a green solid (14.9 mg, 57%). UV/Vis (methanol) λₘₐₓ=787 nm; LRMS (ES/acetonitrile), m/z calculated for C₅₉H₇₅N₆O₉S [M+H]⁺1043.5. found 1043.9.

Example 2 illustrates the synthesis of 4-(2-((1E,3E,5E)-5-(3-(1-azido-13-oxo-3,6,9,14-tetraoxa-12-azaicosan-20-yl)-1,1-dimethyl-1H-benzo[e]indol-2(3H)-ylidene)penta-1,3-dienyl)-1,1-dimethyl-1H-benzo[e]indolium-3-yl)butane-1-sulfonate (“Compound 2”).

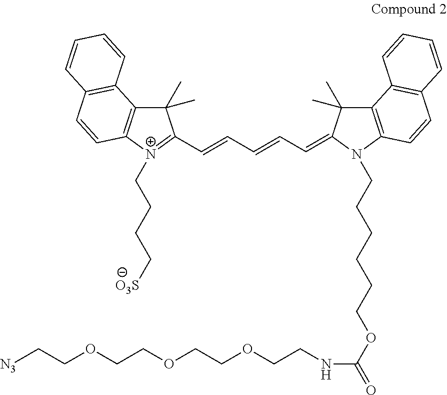

To a vigorously stirred solution of N,N′-disuccinimidyl carbonate (30.0 mg, 0.12 mmol) and N,N-diisopropylethylamine (0.040 mL, 0.24 mmol) in anhydrous acetonitrile (4.0 mL) was added 4-(2-((1E,3E,5E)-5-(3-(6-hydroxyhexyl)-1,1-dimethyl-1H-benzo[e]indol-2(3H)-ylidene)penta-1,3-dienyl)-1,1-dimethyl-1H-benzo[e]indolium-3-yl)butane-1-sulfonate (30.0 mg, 0.043 mmol) in one portion. The reaction was allowed to stir at ambient temperature for 12 hours, during which the presumed mixed carbonate intermediate had formed (as determined by HPLC analysis). A solution of 11-azido-3,6,9-trioxaundecan-1-amine (30.0 mg, 0.14 mmol) in anhydrous acetonitrile (0.5 mL) was added in one portion and the reaction was allowed to stir at ambient temperature for an additional 2 hours. After HPLC analysis showed complete consumption of the presumed mixed carbonate intermediate, the reaction mixture was concentrated in vacuo to afford a crude residue. The residue was purified by reverse-phase flash chromatography to furnish the desired product IR700-PEG-Azide as a blue solid (32.5 mg, 80%). UV/Vis (methanol) λₘₐₓ=680 nm; LRMS (ES/acetonitrile), m/z calculated for C₅₂H₆₅N₆O₈S [M−H]⁻ 933.5. found 933.8.

Example 3 illustrates the synthesis of 2-((1E,3Z,5E)-5-(1,1-dimethyl-6,8-disulfo-3-(3-sulfopropyl)-1H-benzo[e]indol-2(3H)-ylidene)-3-(3-(5-oxo-5-(prop-2-ynylamino)pentyl)phenyl)penta-1,3-dienyl)-1,1-dimethyl-8-sulfo-3-(3-sulfopropyl)-1H-benzo[e]indolium-6-sulfonate (“Compound 3”).

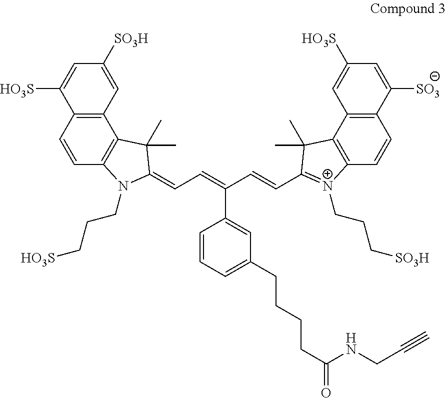

To a vigorously stirred solution of Compound 3 NHS ester (2.5 mg, 1.8×10⁻³ mmol) and N,N-diisopropylethylamine (0.004 mL, 2.3×10⁻² mmol) in anhydrous dimethyl sulfoxide (0.2 mL) was propargylamine hydrochloride (1.0 mg, 1.1×10⁻² mmol) in one portion. After HPLC analysis showed near-complete consumption of Compound 3 NHS ester, the reaction mixture was precipitated into anhydrous diethyl ether. The crude precipitate was purified by reverse-phase HPLC. Fractions of ≧95% product purity by HPLC analysis were combined and lyophilized to afford the desired product Compound 3 as a blue flocculent solid (2.2 mg, 90%). UV/Vis (methanol) λₘₐₓ=677 nm; LRMS (ES/water), m/z calculated for C₅₃H₅₈N₃O₁₉S₆ [M+H]⁺ 1232.2. found 1232.4.

Example 4 illustrates the synthesis of 2-((1E,3Z,5E)-3-(3-(5-((1-(20-((E)-2-((E)-2-(3-((E)-2-(3,3-dimethyl-1-(4-sulfonatobutyl)-3H-indolium-2-yl)vinyl)-2-phenoxycyclohex-2-enylidene)ethylidene)-1,1-dimethyl-1H-benzo[e]indol-3(2H)-yl)-13-oxo-3,6,9,14-tetraoxa-12-azaicosyl)-1H-1,2,3-triazol-4-yl)methylamino)-5-oxopentyl)phenyl)-5-(1,1-dimethyl-6,8-disulfo-3-(3-sulfopropyl)-1H-benzo[e]indol-2(3H)-ylidene)penta-1,3-dienyl)-1,1-dimethyl-8-sulfo-3-(3-sulfopropyl)-1H-benzo[e]indolium-6-sulfonate (“Compound 4”).

Click Solution:

The Click Solution was prepared by adapting procedures prescribed by pertinent references in Section 3. Copper(I) iodide (1.0 mg, 5.3×10⁻³ mmol) was dissolved in 0.11 mL of dimethyl sulfoxide. TBTA (6.0 mg, 1.1×10⁻² mmol) was added in one portion. The resulting turbid solution was vortexed thoroughly for 1 minute and shielded from light until use.

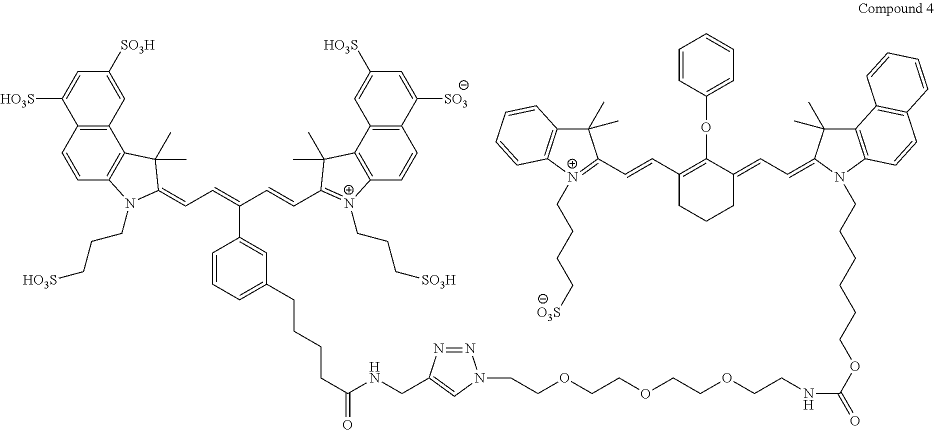

To a solution of Compound 1 (52 μg, 0.050 μmol) in dimethyl sulfoxide (10 μL) was added a solution of Compound 3 (34 μg, 0.023 μmol) in water (20 μL) followed by the aforementioned Click Solution (10 μL). The reaction was allowed to proceed at ambient temperature for 2 hours, with periodic agitation every 15 minutes. After HPLC analysis showed near-complete consumption of Compound 3, the reaction mixture was diluted with methanol (100 μL) and filtered to remove particulates. The filtrate was subjected to reverse-phase HPLC purification; fractions containing the Compound 4 (“Click Product”) were collected and concentrated in vacuo. UV/Vis (methanol) λₘₐₓ1=789, λₘₐₓ2=679 nm; LRMS (ES/water), m/z calculated for C₁₁₂H₁₁₂N₉O₂₈S₇ [M−2H]²⁻, 1136.4. found 1136.1. The deconvoluted mass (M=2274.8) matched the predicted exact mass of the desired IR800-IRDye 680LTClick Product.

Example 5 illustrates the synthesis of 2-((1E,3Z,5E)-3-(3-(5-((1-(20-((E)-2-((2E,4E)-5-(1,1-dimethyl-3-(4-sulfonatobutyl)-1H-benzo[e]indolium-2-yl)penta-2,4-dienylidene)-1,1-dimethyl-1H-benzo[e]indol-3(2H)-yl)-13-oxo-3,6,9,14-tetraoxa-12-azaicosyl)-1H-1,2,3-triazol-4-yl)methylamino)-5-oxopentyl)phenyl)-5-(1,1-dimethyl-6,8-disulfo-3-(3-sulfopropyl)-1H-benzo[e]indol-2(3H)-ylidene)penta-1,3-dienyl)-1,1-dimethyl-8-sulfo-3-(3-sulfopropyl)-1H-benzo[e]indolium-6-sulfonate (“Compound 5”).

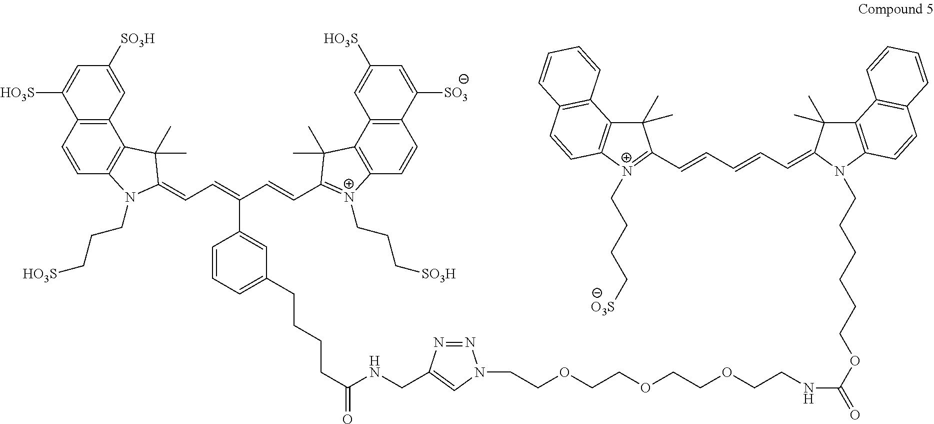

To a solution of Compound 2 (47 μg, 0.050 μmol) in dimethyl sulfoxide (10 μL) was added a solution of Compound 3 (34 n, 0.023 μmol) in water (20 μL) followed by the aforementioned Click Solution (10 μL). The reaction was allowed to proceed at ambient temperature for 2 hours, with periodic agitation every 15 minutes. After HPLC analysis showed near-complete consumption of Compound 3, the reaction mixture was diluted with methanol (100 μL) and filtered to remove particulates. The filtrate was subjected to reverse-phase HPLC purification; fractions containing the presumed Compound 5 were collected and concentrated in vacuo. UV/Vis (methanol) λₘₐₓ=679 nm; LRMS (ES/water), m/z calculated for C₁₀₅H₁₂₁N₉O₂₇S₇[M−2H]²⁻ 1082.3. found 1082.6. The deconvoluted mass (M=2164.6) matched the predicted exact mass of the desired Compound 5.

All publications, patents and patent applications cited in this specification are herein incorporated by reference as if each individual publication, patent or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound of formula I: 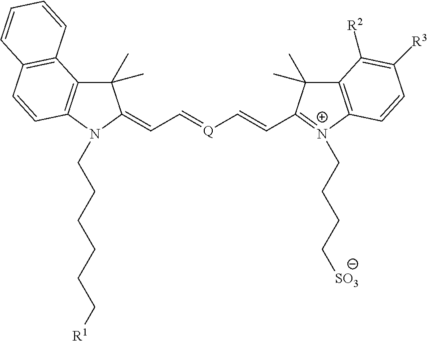
wherein R¹ is a member selected from the group consisting of an azido, a linker having an azido moiety, C₂₋alkynyl, a linker having a C₂₋alkynyl, a pegylated azido and a pegylated C₂₋alkynyl;
wherein R² and R³ are the same or different and wherein each is a member selected from the group consisting of H, cyano, halo, trifluoromethylsulfonyl, sulfonato, or alternatively, join to form a six membered ring;
Q, if present is 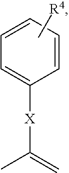
wherein X is a bond or heteroatom; and
R⁴ is a member selected from the group consisting of an azido, C₂₋alkynyl, a linker having a C₂₋alkynyl, a pegylated azido and a pegylated C₂₋alkynyl.

2. The compound of claim 1, wherein Q is absent and R² and R³ join to form a six membered ring.

3. The compound of claim 2, said compound having formula:

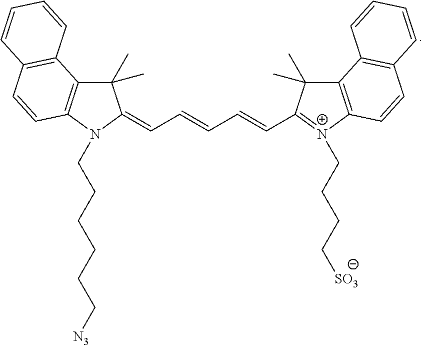

4. The compound of claim 2, said compound having formula:

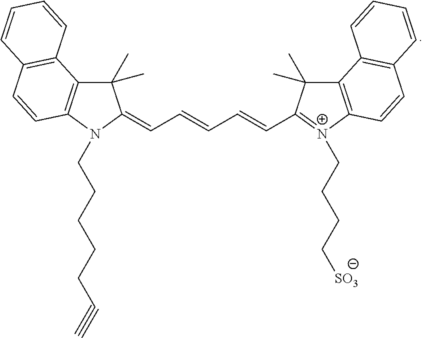

5. The compound of claim 2, wherein R¹ is a member selected from the group consisting of a pegylated azido and a pegylated C₂₋alkyne.

6. The compound of claim 5, said compound having formula:

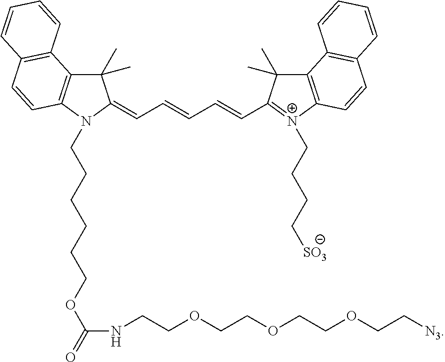

7. The compound of claim 1, wherein Q is present.

8. The compound of claim 7, wherein X is oxygen.

9. The compound of claim 8, said compound having formula:

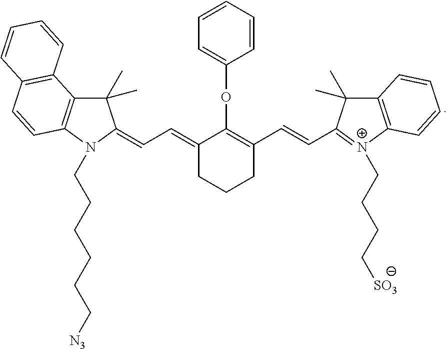

10. The compound of claim 7, said compound having formula:

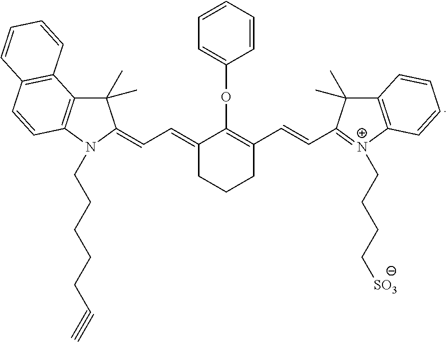

11. The compound of claim 7, wherein R¹ is a member selected from the group consisting of a pegylated azido and a pegylated C₂₋alkyne

12. The compound of claim 11, said compound having the formula:

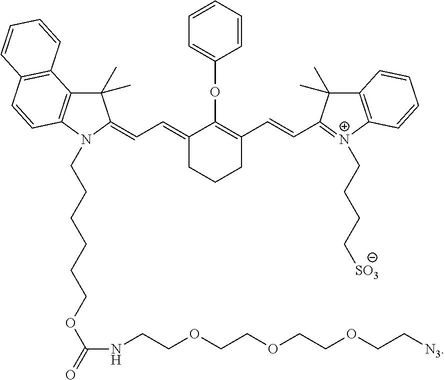

13. A compound selected from the group consisting of a compound having Formula IIa: 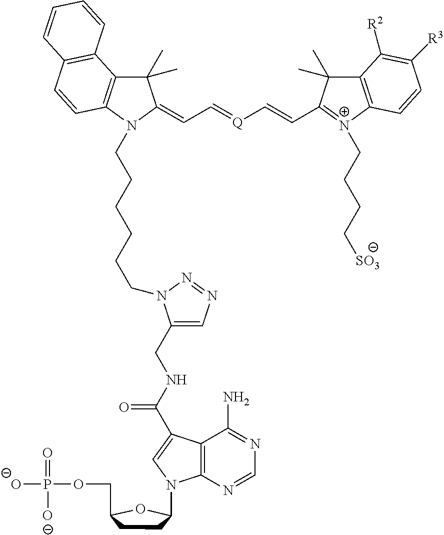
wherein R² and R³ are the same or different and wherein each is a member selected from the group consisting of H, cyano, halo, trifluoromethylsulfonyl, sulfonato, or alternatively, join to form a six membered ring; a compound having Formula IIb: 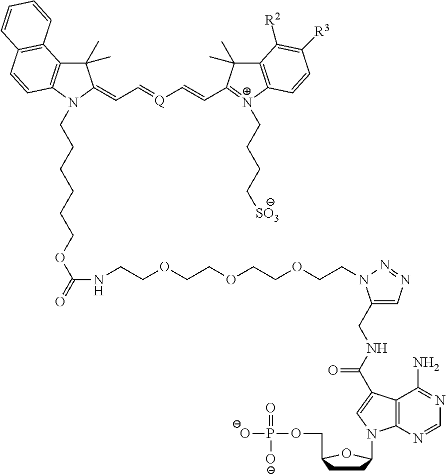
wherein R² and R³ are the same or different and wherein each is a member selected from the group consisting of H, cyano, halo, trifluoromethylsulfonyl, sulfonato, or alternatively, join to form a six membered ring; a compound having Formula IIIa: 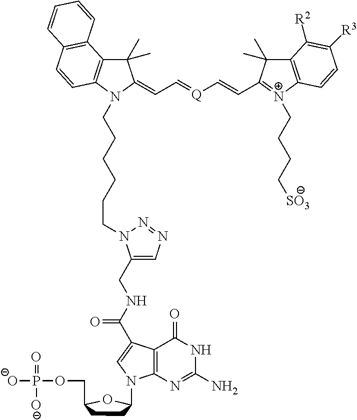
wherein R² and R³ are the same or different and wherein each is a member selected from the group consisting of H, cyano, halo, trifluoromethylsulfonyl, sulfonato, or alternatively, join to form a six membered ring; a compound having Formula IIIb: 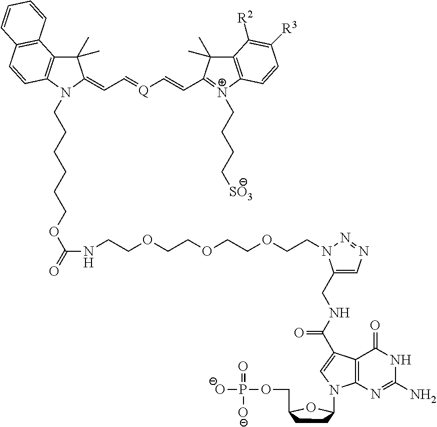
wherein R² and R³ are the same or different and wherein each is a member selected from the group consisting of H, cyano, halo, trifluoromethylsulfonyl, sulfonato, or alternatively, join to form a six membered ring; a compound having Formula IVa: 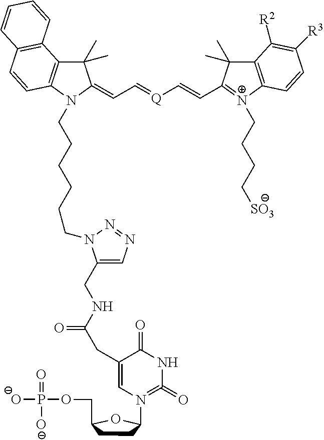
wherein R² and R³ are the same or different and wherein each is a member selected from the group consisting of H, cyano, halo, trifluoromethylsulfonyl, sulfonato, or alternatively, join to form a six membered ring; a compound having Formula IVb: 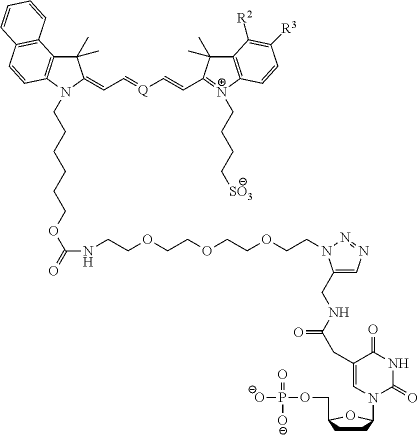
wherein R² and R³ are the same or different and wherein each is a member selected from the group consisting of H, cyano, halo, trifluoromethylsulfonyl, sulfonato, or alternatively, join to form a six membered ring; a compound having Formula Va: 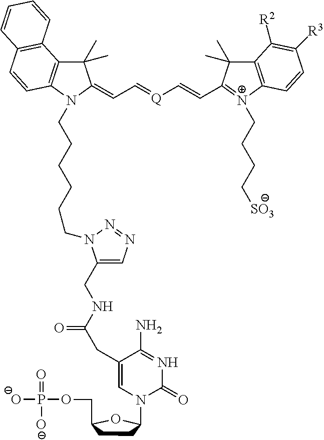
wherein R² and R³ are the same or different and wherein each is a member selected from the group consisting of H, cyano, halo, trifluoromethylsulfonyl, sulfonato, or alternatively, join to form a six membered ring; and a compound having Formula Vb: 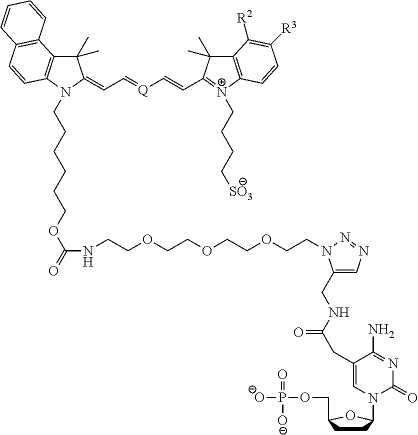
wherein R² and R³ are the same or different and wherein each is a member selected from the group consisting of H, cyano, halo, trifluoromethylsulfonyl, sulfonato, or alternatively, join to form a six membered ring.

14. A method for nucleic acid synthesis, said method comprising:
reacting a polymerase, a nucleic acid template, a primer, dNTPs, and other reagents necessary for polymerization; and
incorporating a chain terminator selected from the group consisting of Formulas IIa-b, IIIa-b, IVa-b, Va-b and mixtures thereof, whereby nucleic acid synthesis ceases.
